(19) United States
(12) Patent Application Publication
(10) Pub. No.: US 2004/0037751 A1
(43) Pub. Date: Feb. 26, 2004

(54) Electromagnetic control of pathogens in poultry production facilities

(76) Inventors: J. Robert Wayland, Albuquerque, NM (US); Philip Baugh, Moticello, AR (US); Philip Moore, Fayetteville, AR (US)

Correspondence Address: John E. Halamka, Esq., P.O. Box 207, Palos Verdes Estates, CA, 90274, US

(21) Appl. No.: 10/189,730

(22) Filed: Jul. 3, 2002

(51) Int. Cl. A61L 9/00

(52) U.S. Cl. 422/121; 422/1; 422/4

(57) ABSTRACT

Electromagnetic energy the microwave range, radio frequency range or a combination of both frequencies may be used to treat poultry litter or other animal wastes to remove or reduce pathogens such as bacteria, viruses, fungi, etc. that affect animal and human health. The treatment may be applied to the litter as well as to the floor upon which the litter is setting. The preselected amount and type of energy applied by radio frequency and microwave frequency depends upon the conditions of the litter and floor such as thickness, material, moisture content and length and of intensity of use of the litter. The litter may be treated in place on the floor to avoid contamination caused by moving the litter from the floor and into a treatment chamber.

### BACKGROUND OF THE INVENTION

This invention relates to the method and apparatus for the treatment of poultry litter and other animal wastes to reduce the number of pathogens (bacteria, viruses, fungi, etc.) that affect animal and/or human health and wellbeing. More specifically to the in place treatment of poultry litter by the use of electromagnetic energy in the frequency range of microwave, radio frequency or both to remove and/or reduce pathogens in the litter. Also to the use of the above mentioned electromagnetic energies to batches of litter placed in a chamber to remove and/or reduce pathogens.

Poultry and other livestock are commonly reared in confinement on a bedding material. The bedding material can consist of wood shavings, sawdust, rice hulls, peanut hulls, straw and recycled paper products in different mixtures and compositions. When animals are reared on this material the resultant combination of manure, bedding material, feathers, etc., is referred to as “litter”. If broilers (meat chickens) are being grown, then broiler litter is produced. Similarly, if turkeys are being produced, then turkey litter is produced.

In a typical production facility about five or six flocks are grown each year on the same litter. In the spring the litter is cleaned out and applied to land as fertilizer, fresh bedding is placed into the houses, and the cycle is repeated. This reuse of the litter leads to a buildup of high levels of bacteria, viruses and fungi. These pathogens, such as E. coli, Salmonella, Clostridium, and Camplyebacter, are not only detrimental to the health and well being of the birds, many of them also pose a significant risk to human health. Food borne illness is a major problem in the U.S., particularly with poultry, and there are very few methods for controlling pathogen levels in poultry litter.

The litter may be buried, but this results in the possibility of contamination of the environs and ground water supplies. Steam sterilization of the litter before further processing is expensive and time consuming. The monitoring of the process for effectiveness is difficult and expensive. Dodd in U.S. Pat. No. 2,731,208 teaches a method of treating medical waste by shredding the waste and blowing steam into a container full of the waste. This is effective only for limited volumes and has an additional complication of depositing the shredded mixture into a sewer.

There are systems for treating waste utilizing microwaves. One system described in The Wall Street Journal (pg. B33, Apr. 10, 1989) picks up the waste, shreds it, sprays it with water and exposes it to microwaves in a chamber long enough to raise the temperature to 205 degrees C. This approach will not work for poultry litter because of the need to move the litter into an exposure chamber an then depositing it back into the beds. This increases the exposure to infectious particles and is expensive.

Kartchner in U.S. Pat. No. 5,980,824 describes a device to apply microwaves to animal waste. The device taught in this patent suffers from the same restriction as above.

Microwaves are limited in their penetration but are highly effective in the sterilization of the poultry litter. Their use has the advantage of control without leaving harmful residues or causing environmental pollution. There remains the problem of a contaminated floor that can re-infest the litter. Thus, there is a need for treatment of the floor that has the same advantage as the microwave treatment of the litter.

Wayland in U.S. Pat. No. 5,060,414 (Oct. 29, 1991) teaches the use of multiple frequencies in the control of unwanted vegetation. The underling concept is that, by combining in a specific application of multiple frequency electromagnetic fields, the destructive physiological response will be enhanced. This invention does not depend upon this effect. The use of multiple frequencies in this invention is for the purpose of insuring that sufficient electromagnetic energy will be deposited in each segment of a litter bed to maintain control of unwanted pathogens.

### SUMMARY OF THE INVENTION

Accordingly, it is an object of the present invention to provide a new and improved method of and apparatus for the reduction and control of unwanted pathogens in poultry litter and in the floor. To insure the necessary control in both the litter and the floor, two or more different frequencies of electromagnetic energy are used. At least one frequency in the microwave frequency band and another in the radio frequency band. A method and apparatus will be described that may be tuned to provide a preferred mixture of frequencies and energy levels for the reduction and control of unwanted pathogens in poultry litter and in the floor.

The control is achieved by two or more sets of transmitters and radiators, one in the radio frequency spectrum and at least a second in the microwave region. The radiators are set to apply directly, and in close distance, the electromagnetic field to the poultry litter. They are mounted on a cart or vehicle. The power levels and duration of exposure are adjustable by the user to accommodate particular litter configuration and conditions. This application teaches that by using a combination of frequencies, treatment of both the litter and its floor can provide the reduction and control of unwanted pathogens in poultry litter. Depending upon the condition of the litter and floor, the frequencies chosen will be optimize to produce the most effective treatment. Thus, there maybe two, three or more frequencies applied in any given treatment.

Additional aspects and advantages of the invention will be set forth in part of the description that follows, and in part will be obvious from the description, or may be learned by practice of the invention. The advantages of the invention will be realized and attained by means of the elements and combinations particularly pointed out in the appended claims. It is understood that both the foregoing general description and the followed detailed description are exemplary and explanatory only and are not restrictive of the invention, as claimed.

### DESCRIPTION OF THE INVENTION

The above and other embodiments of the present invention may be more fully understood from the following description and accompanying drawings.

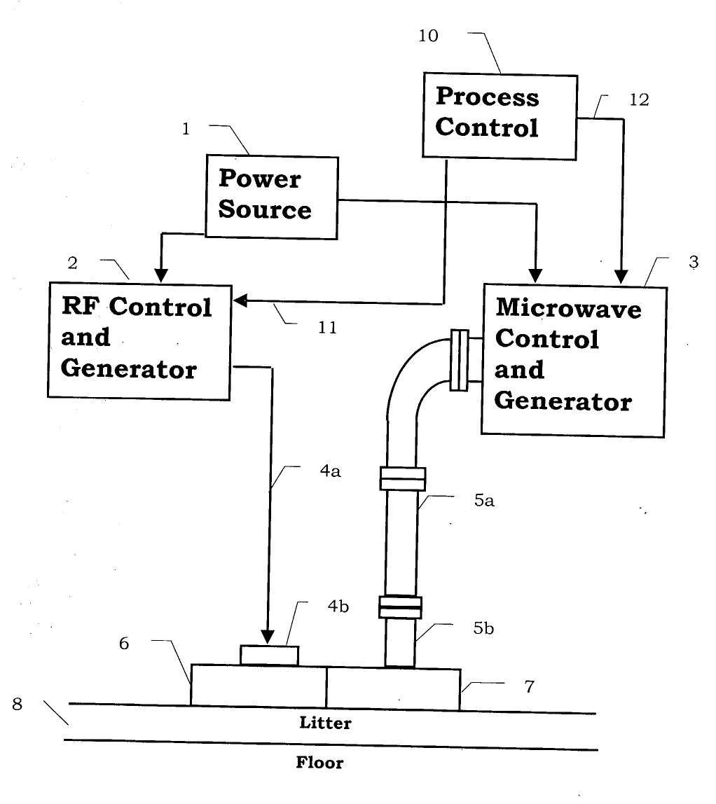

FIG. 1 block diagram of a device that performs the method of applying at least two electromagnetic fields to poultry litter;

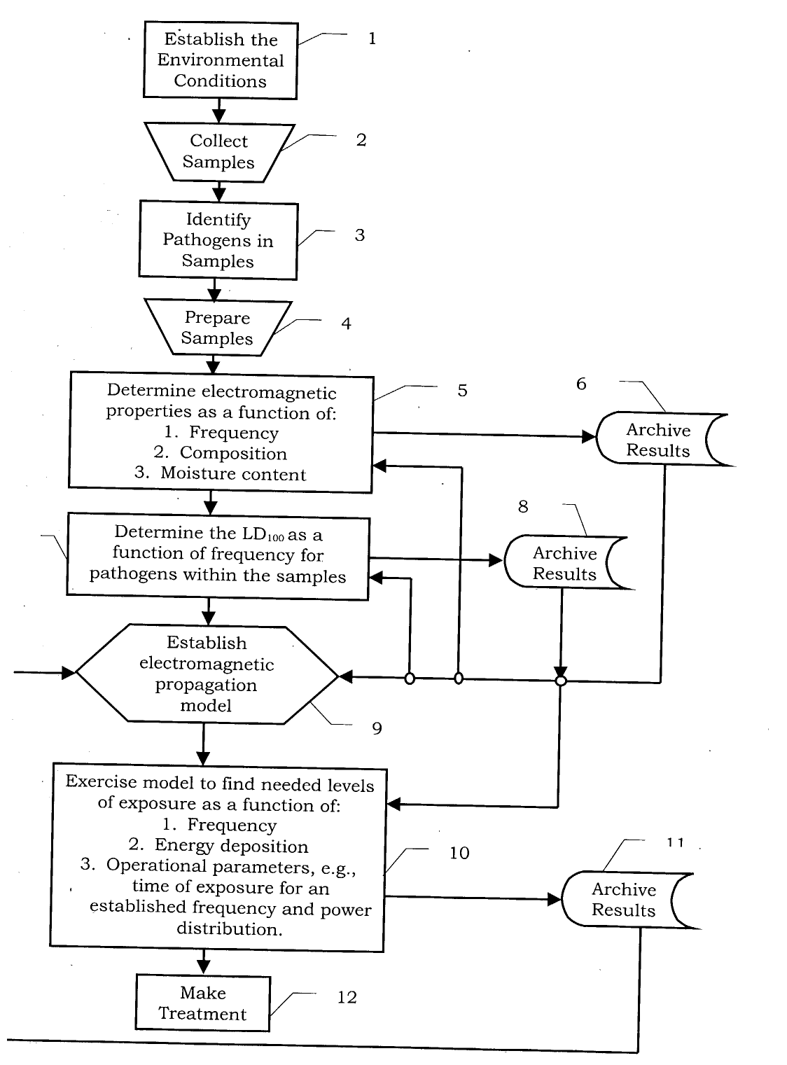

FIG. 2 flow diagram of the analysis for the proper configuration of the electromagnetic field applicator.

Referring to the drawing FIG. 1, a diagrammatic view illustrating the electromagnetic field generating equipment, 2 and 3, the radiating elements, 6 and 7, and the process control network, 10 for effective control of the application according to the teaching of this invention.

The products built according to the teachings of this invention may utilize standard radio frequency networks and devices, and standard microwave networks and devices well known in the art shown in FIG. 1.

The microwave (MW) electromagnetic fields (EMF) may be generated by means of magnetrons. The control and transmission of the energy and coupling into a radiator are well known in the art. It is critical that good safety practices are followed. The radio frequency (RF) EMF can be generated using standard commercial equipment. Depending upon the RF radiator employed, the RF EMF may be radiated as a uniform EMF over relative large areas. However, the MW EMF is highly directional and the radiator must be designed carefully to apply the MW EMF directly into the litter/floor media. The depth of penetration into the litter/floor is a function of wavelength and the electromagnetic properties of the litter/floor. The moisture content of the litter/floor is an important variable in determining the distribution of absorbed energy in the litter/floor. This invention teaches that the frequencies and radiation patterns of the applied EMFs should be chosen to optimize the absorption of energy so that the control of pathogens is maximized. Depending upon the conditions in the litter/floor, it may be important to deposit more EMF energy in either the litter or the floor.

To accomplish the best distribution of energy in the litter/floor a process similar to the flow chart shown in FIG. 2 should be followed. Step 1, will be to establish the environmental conditions in the production facility. These will include such data as the layout, the thickness of the litter, moisture content of the litter and of the floor, etc. Samples of the litter and the floor, as indicated in step 2 and 3, will be assayed to determine what pathogens are present. These samples will then be prepared for testing in a controlled laboratory apparatus for exposure to Electromagnetic fields, step 4. The exposure chambers must have the necessary environmental controls and the ability to make exposures over appropriate frequency ranges and power levels. Then using values from the open literature or from archives, database 6, of previous tests as a starting point, determine, as indicated in step 5, the electromagnetic properties of the samples as a function of frequency, composition, moisture content, etc. These measured values are archived, in database 6, for use in future tests. Simultaneously or after these tests are run, a series of exposures should be made to determine the lethal dose, 7, to kill 95% to 100%, i.e., the LD₁₀₀ of the pathogens. (LD₁₀₀ is the Lethal Dose to kill 100% of the pathogens.) These tests will be guided by the information in database 8. At times this will be an overkill and a lesser kill percentage may suffice. These results are archived in the database 8 for use in future tests as the starting point in the next application.

The next steps, 9 and 10, require the solution of Maxwell's equations. Depending upon the geometry of the radiating element, there are a number of standard analytic solutions (see J. R. Wait, Electromagnetic Waves in Stratified Media, New York: Pergamon, 2^{nd} Edition 1970). It is also possible to obtain numerical solutions of the fundamental equations using computer integration techniques. Again, the results are archived in database 11 for future use. The databases 6, 8 and 11 are the accumulated The results of this calculation will instruct the operator on the proper application of this technique to insure needed control of pathogens in the production facility.

As an example, when the above procedure was applied to a typical poultry raising facility it was found that a frequency of 433 MHz at 50 KWatts/SqFt followed by an application of 2450 MHz at 30 KWatts/SqFt at a speed of 4 Ft/sec would provide good control. Of course, this would not be the same for all applications. Each application would require analysis.

Clearly, after a body of knowledge is accumulated, not all of the above steps will be needed for each application. Only when new and uninvestigated situations are encountered will this entire lengthy procedure be necessary.

The toxicity of radio frequencies and microwave fields to pathogens (bacteria, viruses, fungi, etc.) is well known in the art. There are many possible mechanisms for this result. There is certainly a component associated with the increase in temperature, but there are also non-thermal effects. The actual mechanism of the phenomena is not well understood. Without specifying the precise mechanism it is the teaching of this patent that effective control of pathogens, in poultry litter and the floor upon which the litter rests, can be accomplished by the proper application of a multiple frequency electromagnetic field.

Since certain changes may be made in the above apparatus without departing from the scope of the invention herein involved, it is intended that all matter contained in the above description, as shown in the accompanying drawings, shall be interpreted in an illustrative, and not a limiting sense.

References

Greca et al., “Effect of radiofrequency energy on bacterial spores” Bacteriol. Proc., p.145 (1964); Fleming, P., “Effect of High Frequency Fields on Micro-Organisms,” Elec. Eng., Vol. 63(1), pp. 18-21 (1944); Chipley, J., “Effects of Microwave Irradiation on Microorganisms”, Adv. Appl. Microbio., vol.26, pp. 129-145 (1980); Wait, J. R., “Electromagnetic Waves in Stratified Media”, Pergamon Press, 1970; Kaufman, A. A. and Keller, G. V., “Frequency and Transient Soundings”, Elserier Sci. Pub., 1983: Wait, J. R., “Geo-electromagnetism”, Academic Press, 1982; Lechowich, et al., “Procedure for Evaluating the Effects of 2,450 MHz Microwaves upon Streptococcus faecalis and Saccharomyes cereislae”, Applied Microbiology, vol 17, pp. 106-110, 1969; Wayland, et al., “On the Interdependence of Thermal and Electromagnetic Effects in the Response of Bacillus subtilis Spores to Microwave Exposure, Radiation Research, vol. 71, pp. 251-258 (1977)

What is claimed is:

1. A method of pathogen and disease vectors reduction in animal rearing facilities having a floor covered with animal litter, comprising the steps of:
first, generating a plurality of preselected radio frequency electromagnetic fields at preselected power levels for emission from radiators;
second, generating a plurality of preselected microwave electromagnetic fields at preselected power levels for emission from radiators;
positioning said radiators to expose said animal litter and a preselected portion of the top surface of said floor in animal rearing facilities whereby the application of said microwave and radio frequency electromagnetic fields for a preselected duration to produce death or debilitation of the pathogen and disease vectors.

2. A method of pathogen and disease vectors reduction in animal rearing facilities defined in claim 1 wherein said generated electromagnetic fields are pulsed waves.

3. A method of pathogen and disease vectors reduction in animal rearing facilities defined in claim 1 wherein said generated electromagnetic fields are continuous waves.

4. A method of pathogen and disease vectors reduction in animal rearing facilities defined in claim 1 wherein said generated electromagnetic fields are modulated waves.

5. A method of pathogen and disease vectors reduction in animal rearing facilities defined in claim 1 wherein said generated electromagnetic fields are combination of continuous, modulated and pulsed waves.

6. An improved electromagnetic field generation and application arrangement for pathogen and disease vectors reduction in animal rearing facilities having a floor covered with animal litter, comprising, in combination:
a means for generating a radio frequency electromagnetic field;
a means for generating a microwave electromagnetic field;
a radio frequency radiator connected to said radio frequency generation means;
a microwave radiator connected to said microwave generation means;
means for applying said electromagnetic radiation to said animal litter and floor under the conditions of bringing said radiator in proximity to said animal litter and floor whereby causing the application of said electromagnetic fields for a preselected duration.

7. The arrangement defined in claim 6 wherein said means to apply is a vehicle.

8. The arrangement defined in claim 6 wherein said means to apply is a conveyer belt to guide said animal litter into the proximity of said radiators.

9. An improved electromagnetic field generation and application arrangement for pathogen and disease vectors reduction in animal rearing facilities comprising in combination:
a means of establishing the frequency dependent energy absorption needed for reduction of specific pathogens either from the open literature or from laboratory studies;
a means of calculation of the frequency dependent propagation of electromagnetic waves in the layered media of the litter and floor;
a means of calculation of the frequencies and radiation patterns of the applied EMFs that should be chosen to optimize the absorption of energy;
using this information to determine the frequencies, power levels and exposure duration so that the control of pathogens is maximized.

10. Using the arrangements specified in claims 1 through 7 and the information developed in claim 9 to produce death or debilitation of the pathogen and disease vectors in animal rearing facilities.
